(19) United States
(12) Patent Application Publication
(10) Pub. No.: US 2016/0317727 A1
(43) Pub. Date: Nov. 3, 2016

(54) Blood Bag System and Tube Holder

(71) Applicants: TERUMO BCT, INC., Lakewood, CO (US); TERUMO KABUSHIKI KAISHA, Tokyo (JP)

(72) Inventors: Makoto HIRABUKI, Shizuoka (JP); Tadashi IMAI, Leuven (BE)

(73) Assignees: Terumo BCT, Inc., Lakewood, CO (US); Terumo Kabushiki Kaisha, Tokyo (JP)

(21) Appl. No.: 15/108,611

(22) PCT Filed: Nov. 19, 2014
(86) PCT No.: PCT/US2014/066358

§ 371 Date: Jun. 28, 2016

(30) Foreign Application Priority Data
Nov. 29, 2013 (JP) 2013-246998

(51) Int. Cl. A61M 1/02; B04B 5/04; A61M 1/36

(52) U.S. Cl. CPC A61M 1/0218; A61M 1/3693; A61M 1/3696; B04B 5/0428; A61M 2205/12; B04B 2005/0435

(57) ABSTRACT

In order to prevent or suppress a blood component from being unnecessarily mixed to other bags by returning blood moved to a tube from a bag before centrifuge to the bag during the centrifuge. A blood bag system (10) has a cassette (50) (tube holder) for holding at least a part of the first transfer tube (21). The cassette (50) holds the first transfer tube (21) such that a distance between a rotation center (O) in application of a centrifugal force and the first transfer tube (21) increases toward the blood collection bag (11) side across a length between a flow path close position caused by the first clamp (71) in the first transfer tube (21) and the blood collection bag (11).

### TECHNICAL FIELD

The present invention relates to a blood bag system configured to transfer components obtained by centrifuging whole blood or blood components from a bag to an outer side of the bag through a tube held by a tube holder and a tube holder applicable to such a blood bag system.

### BACKGROUND ART

In recent technologies of blood transfusion, blood component transfusion is employed, in which components of blood (whole blood) obtained from blood donation and the like are separated, and only a component necessary for a patient is supplied. The blood component transfusion makes it possible to reduce burdens or side effects on a patient's circulatory system and effectively use the donated blood.

In the separation of the blood components, a blood bag system having a plurality of bags and a plurality of tubes is installed in a centrifuge/transfer apparatus, for example, as disclosed in Patent Document 1. In addition, the centrifuge/transfer apparatus centrifuges blood into a light supernatant PPP (platelet poor plasma) fraction, a heavy precipitated RCC (concentrated red blood cells) fraction, and a buffy coat formed therebetween. Each centrifuged component is transferred to and preserved in a plurality of bags.

However, if the blood bag system is directly installed in the centrifuge/transfer apparatus as described above, a plurality of bags or a plurality of tubes are complicatedly entangled, so that installation becomes difficult, and workability is degraded. In this regard, a blood bag system (for example, as discussed in Patent Document 2 described below) has been proposed, in which a blood collection bag that stores whole blood and a part of tubes connected to this blood collection bag are fixedly held in a cassette that serves as a tube holder. In this blood bag system, since the blood bag system is installed in the centrifuge/transfer apparatus using the cassette, a work load necessary in wiring of tubes and the like is remarkably reduced, and workability is improved.

### CITATION LIST

### Patent Document

Patent Document 1: U.S. Pat. No. 6,910,998

Patent Document 2: JP 2012-510298 W

### SUMMARY OF THE INVENTION

### Problems to be Solved by the Invention

Such a blood collection bag of the blood bag system having a cassette described above internally contains an additive solution (for example, a blood preservation solution having a blood anticoagulant capability) in advance. For this reason, if a tube connected to the blood collection bag for transferring plasma communicates with this blood collection bag in an initial state, apart of the additive solution inside the blood collection bag is input to the tube before collection of blood (whole blood) into the blood bag. As a result, a part of blood of the blood collection bag may diffuse to an additive solution of the tube after collection of blood and may move to the inside of the tube. For this reason, in a process of transferring plasma to the plasma bag after the centrifuge, a small amount of blood moved to the inside of the tube (including red blood cells) may be mixed into the plasma bag. Since a small amount of the red blood cells are mixed into the plasma bag, there may be no problem in quality as a plasma derivative, but this is not preferable in terms of appearance.

In view of such problems, it is an object of this invention to provide a blood bag system capable of preventing or suppressing unnecessary blood components from being mixed to other bags by returning blood moved to the inside of the tube from the bag before centrifuge to the bag during the centrifuge, and a tube holder applicable to such a blood bag system.

### Solutions to Problems

In order to achieve the aforementioned objects, according to an aspect of the invention, there is provided a blood bag system including: a bag configured to preserve whole blood or a blood component; a tube configured to transfer a component obtained by centrifuging a content of the bag to an outer side of the bag; and a tube holder configured to hold at least a part of the tube, wherein the tube is configured to close and open a flow path while the tube is held by the tube holder, and the tube holder holds the tube such that a distance between a rotation center in application of a centrifugal force and the tube increases toward the bag side across a length between a flow path close position of the tube and the bag.

In this configuration, even when a part of blood inside the bag after collection of blood moves to the inside of the tube, the blood input to the tube is almost or perfectly returned to the bag by virtue of a centrifugal force during the centrifuge process. Therefore, in a process of transferring a predetermined blood component obtained through the centrifuge to another bag, it is possible to prevent or suppress unnecessary blood cells from being mixed to another bag. Therefore, it is possible to manufacture blood component derivatives having an excellent appearance quality.

In the blood bag system described above, the tube holder may hold the tube such that the tube has a first portion that extends in a centrifugal direction and is connected to the bag and a second portion that extends perpendicularly to the centrifugal direction and reaches the flow path close position. In this configuration, it is possible to prevent or suppress blood cells from being mixed to the bag for storing a predetermined blood component as described above. In addition, it is possible to hold the tube with a reasonable shape in terms of a relationship with the flow path close position or the holding position of the bag.

In the blood bag system described above, the second portion may have a straight line shape. In this configuration, it is possible to simplify a structure of the portion for holding the tube in the tube holder and facilitate installation of the tube to the tube holder.

In the blood bag system described above, the second portion may be curved or bent. In this configuration, it is possible to return, to the bag, the blood input to the tube in the centrifuge process.

In the blood bag system described above, the bag may include a first bag configured to preserve whole blood or a blood component and a second bag configured to store the component transferred through the tube. In this configuration, it is possible to rapidly advance to a process of transferring a predetermined blood component obtained through the centrifuge to the second bag after the centrifuge of the blood stored in the first bag.

In the blood bag system described above, the component transferred from the bag to an outer side of the bag may be plasma. In this configuration, it is possible to manufacture plasma derivatives with an excellent appearance quality.

According to another aspect of the invention, there is provided a tube holder having a guide path configured to hold at least apart of a tube for transferring a component obtained by centrifuging a content of a bag to an outer side of the bag, wherein the tube is configured to close and open a flow path while the tube is held by the tube holder, and the guide path holds the tube such that a distance between a rotation center of a centrifugal force and the tube increases toward the bag side across a length from a flow path close position of the tube and the bag.

### Effects of the Invention

In the blood bag system and the tube holder according to this invention, it is possible to prevent or suppress an unnecessary blood component from being mixed to other bags by returning the blood moved from the bag to the tube before the centrifuge to the bag during the centrifuge.

### BRIEF DESCRIPTION OF DRAWINGS

FIG. 1 is a schematic diagram illustrating a configuration of the entire blood bag system according to an embodiment of this invention.

FIG. 2 is an exploded perspective view illustrating a cassette.

FIG. 3 is a schematic plan view illustrating a cassette, and a first transfer tube and a blood collection bag held in the cassette.

FIG. 4 is a perspective view illustrating a centrifuge/transfer apparatus.

FIG. 5 is an exploded perspective view illustrating an insert unit.

FIG. 6 is a schematic diagram illustrating a state of the blood bag system before a centrifuge process.

FIG. 7 is a schematic diagram illustrating a state of the blood bag system before a separation/transfer process after the centrifuge process.

FIG. 8 is a schematic diagram illustrating a state of the blood bag system in the course of the separation/transfer process.

### MODE FOR CARRYING OUT THE INVENTION

Hereinafter, a blood bag system and a tube holder according to a preferable embodiment of the invention will be described with reference to the accompanying drawings.

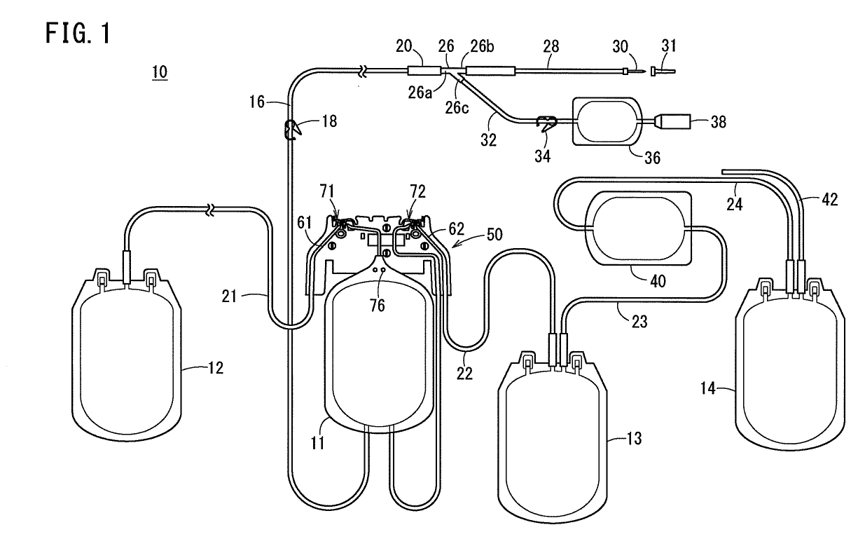

FIG. 1 is a schematic diagram illustrating a configuration of the entire blood bag system 10 according to an embodiment of the invention. The blood bag system 10 is configured to centrifuge blood containing a plurality of components into three components including a low specific gravity component, an intermediate specific gravity component, and a high specific gravity component (in this embodiment, whole blood is centrifuged into plasma, buffy coat (BC), and concentrated red blood cells) and store and preserve each component in different bags. As illustrated in FIG. 1, the blood bag system 10 includes a plurality of bags (first to fourth bags 11 to 14) and a plurality of transfer tubes (first to fourth transfer tubes 21 to 24).

Each of the first to fourth bags 11 to 14 is configured in a bag shape by overlapping flexible sheet materials made of a flexible resin such as polyvinyl chloride or polyolefin on each other and welding or adhering (through thermal fusion bonding or high-frequency fusion bonding) peripheral sealing portions of the sheet materials to each other. Similarly, an initial flow blood bag 36 described below also has a bag shape.

The first bag 11 is a bag for storing (preserving) blood (whole blood) obtained from a donor. Hereinafter, the first bag 11 will be referred to as a “blood collection bag 11.” The blood collection bag 11 is used to store whole blood during blood collection. However, as described below, after the plasma obtained by centrifuging whole blood is transferred to the second bag 12, and concentrated red blood cells are transferred to the third bag 13, the blood collection bag 11 is used to store and preserve the buffy coat which is a residual component. That is, the blood collection bag 11 serves as both a whole blood storing bag and a buffy coat storing bag.

A blood preservation solution having a blood anticoagulant property is stored in the blood collection bag 11 in advance. The blood preservation solution may contain, for example, citric acid, phosphoric acid, and glucose (citrate phosphate dextrose: CPD). The amount of the blood preservation solution is set to an appropriate amount depending on an estimated blood collection amount.

In addition, one end of the blood collection tube 16 in the base end side is connected to the blood collection bag 11. A clamp 18 for opening and closing a flow path of the blood collection tube 16 is provided in the middle of the blood collection tube 16. One end of the seal member 20 is connected to the other end of the blood collection tube 16. The seal member 20 is configured to open the flow path by a breaking manipulation while the flow path is closed in an initial state.

A first port 26a of the branching connector 26 is connected to the other end of the seal member 20. One end of the blood collection tube 28 in the leading edge side is connected to a second port 26b of the branching connector 26. A blood collection needle 30 is connected to the other end of the blood collection tube 28. A cap 31 is installed in the blood collection needle 30 before use.

One end of the branching tube 32 is connected to a third port 26c of the branching connector 26. A clamp 34 for opening and closing a flow path of the branching tube 32 is provided in the middle of the branching tube 32. An initial flow blood bag 36 is connected to the other end of the branching tube 32. A sampling port 38 is connected to the initial flow blood bag 36. It is noted that an orientation or arrangement of the branching connector 26 is not limited to that of FIG. 1, but may be appropriately modified.

A second bag 12 is a bag connected to the blood collection bag 11 through a first transfer tube 21 (tube) to store (keep) and preserve plasma as a low specific gravity component obtained through the centrifuge. Hereinafter, the second bag 12 will be referred to as a “plasma bag 12.” The first transfer tube 21 is a tube for connecting an upper portion of the blood collection bag 11 and an inlet hole of the plasma bag 12 to transfer plasma from the blood collection bag 11 to the plasma bag 12.

The third bag 13 is a bag connected to the blood collection bag 11 through the second transfer tube 22 to store (keep) and preserve concentrated red blood cells as a high specific gravity component obtained through the centrifuge. The second transfer tube 22 is a tube for connecting the blood collection bag 11 and the third bag 13 to transfer concentrated red blood cells from the blood collection bag 11 to the third bag 13. The concentrated red blood cells stored and preserved in the third bag 13 are concentrated red blood cells before removal (filtering) of a predetermined cell (leukocyte).

The third bag 13 stores, for example, a SAG-M (saline adenine glucose mannitol) solution, which is a mixture solution containing mannitol, glucose, adenine, and sodium chloride, as a red blood cell preservation liquid. Therefore, as the concentrated red blood cells obtained through the centrifuge are transferred to the third bag 13, the third bag 13 stores a mixture liquid (hereinafter, referred to as an “RC-SAGM”) of the concentrated red blood cells not subject to filtering and the red blood cell preservation liquid.

The fourth bag 14 is a bag connected to the third bag 13 through the third transfer tube 23, the filter 40, and the fourth transfer tube 24 to store (keep) and preserve concentrated red blood cells with leukocyte being removed (specifically, obtained by removing leukocyte from RC-SAGM).

The third transfer tube 23 is a tube for connecting the third bag 13 and the filter 40 to transfer concentrated red blood cells from the third bag 13 to the filter 40. The filter 40 has a function of removing corpuscles and serves as a leukocyte removing filter in this embodiment.

The fourth transfer tube 24 is a tube for connecting the filter 40 and the fourth bag 14 to transfer the concentrated red blood cells subjected to leukocyte filtering from the filter 40 to the fourth bag 14. It is noted that a sampling tube 42 for drawing a part of concentrated red blood cells is connected to the fourth bag 14.

It is noted that each tube of the blood bag system 10 is formed a transparent flexible resin.

The blood bag system 10 further includes a cassette 50 as a configuration example of the tube holder. According to this embodiment, the cassette 50 is configured to hold the blood collection bag 11 and a part of each of the first and second transfer tubes 21 and 22.

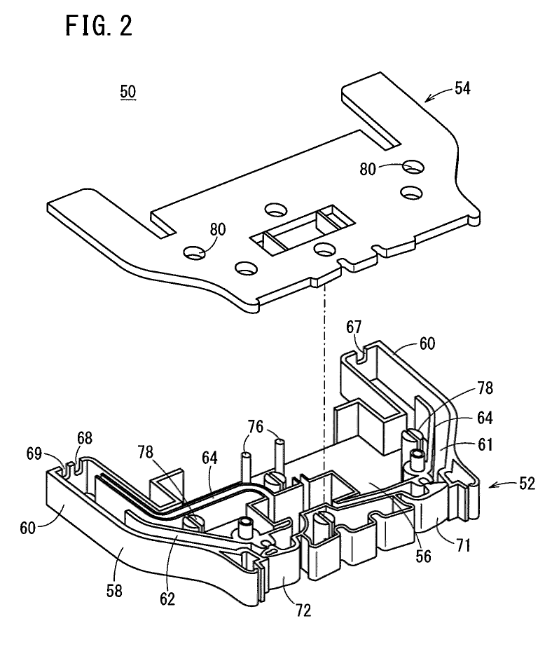

FIG. 2 is an exploded perspective view illustrating the cassette 50. According to this embodiment, the cassette 50 includes a cassette body 52 that supports the blood collection bag 11 and a lid body 54 installed on one side surface of the cassette body 52. The cassette 50 is formed in a substantially flat shape.

The cassette body 52 is formed in a bottomed box shape including a bottom wall 56 and a side wall 58 that surrounds the bottom wall 56. A pair of extensions 60 is protrudingly formed in parallel in both sides of the width direction of the bottom wall 56.

The bottom wall 56 is provided with a first guide path 61 (guide path) capable of fixing and holding the first transfer tube 21 and a second guide path 62 capable of fixing and holding the second transfer tube 22. Both the first and second guide paths 61 and 62 are curved. The first and second guide paths 61 and 62 illustrated in this example are formed by a plurality of walls 64 erected on the bottom wall 56, and a part thereof is formed by the wall 64 and a side wall 58.

It is noted a concave cutout portion 67 for passing the first transfer tube 21 is provided in the leading edge of one of the extensions 60, and a pair of concave cutout portions 68 and 69 for passing the second transfer tube 22 are provided in the leading edge of the other extension 60.

A first clamp 71 for closing and opening a flow path of the first transfer tube 21 is provided in the middle of the first guide path 61. A second clamp 72 for closing and opening a flow path of the second transfer tube 22 is provided in the middle of the second guide path 62.

It is noted that, although the first and second clamps 71 and 72 and the cassette body 52 described in this example are formed in an integrated manner, the first and second clamps 71 and 72 may be formed as an element that is separated from the cassette body 52 and is detachably attached to the cassette body 52.

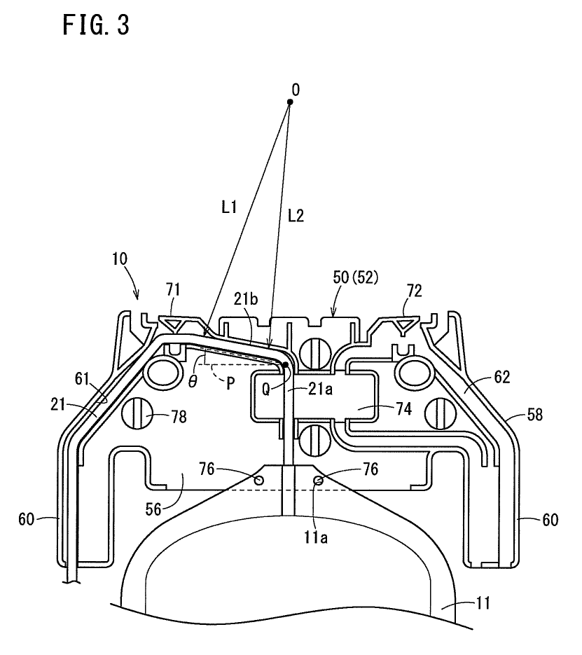

A pair of pins 76 is provided in the center of the bottom wall 56 in the vicinity of the lower end. The pair of pins 76 are inserted into a pair of installation holes 11a (refer to FIG. 3) provided in the upper portion of blood collection bag 11 to hang the blood collection bag 11. A plurality of installation terminals 78 (four installation terminals in this embodiment) connected to the lid body 54 are provided on the front surface of the bottom wall 56.

The lid body 54 is formed in a flat plate shape matching the shape of the cassette body 52. The lid body 54 covers the cassette body 52 during a centrifuge process or a separation/transfer process and protects the first and second transfer tubes 21 and 22 fixedly held in the first and second guide paths 61 and 62. This lid body 54 is preferably formed transparently. As a result, it is possible to recognize a state of the plasma or the concentrated red blood cells flowing through the first and second transfer tubes 21 and 22.

A plurality of installation holes 80 is provided in the lid body 54. As the installation terminals 78 are inserted into and hung on the installation holes 80, the cassette body 52 and the lid body 54 are connected to each other robustly.

FIG. 3 is a schematic plan view illustrating a state that the first transfer tube 21 and the blood collection bag 11 are held by the cassette 50. It is noted that FIG. 3 illustrates a state that the lid body 54 (refer to FIG. 2) is removed from the cassette body 52 in order to focus on a holding state of the first transfer tube 21 using the cassette 50. Although the second transfer tube 22 is also held by the cassette 50 in practical use, this is not illustrated in FIG. 3 intentionally.

As illustrated in FIG. 3, the cassette 50 (the first guide path 61 thereof) holds the first transfer tube 21 such that a distance between a rotation center O in application of a centrifugal force and the first transfer tube 21 increases toward the blood collection bag 11 across a length between a position of the first clamp 71 in the first transfer tube 21 and the blood collection bag 11. In other words, (the first guide path 61 of) the cassette 50 holds the first transfer tube 21 such that a slope is provided in a centrifugal direction toward the blood collection bag 11 side across a length between the position of the first clamp 71 in the first transfer tube 21 and the blood collection bag 11.

According to this embodiment, specifically, the cassette 50 holds the first transfer tube 21 such that the first transfer tube 21 has a first portion 21a that extends in a centrifugal direction during the centrifuge and is connected to the blood collection bag 11 and a second portion 21b that extends perpendicularly to the centrifugal direction and reaches the first clamp 71. A curved portion is interposed between the first and second portions 21a and 21b.

As illustrated in FIG. 3, the second portion 21b extends in a straight line shape. In particular, the second portion 21b does not extend perpendicularly to the first portion 21a, but extends with an inclination to the first portion 21a. This inclination is formed such that the first transfer tube 21 approaches the blood collection bag 11 side from the first clamp 71 side to the first portion 21a side.

An inclination angle of the second portion 21b having a straight line shape with respect to a perpendicular line P against the centrifugal direction during the centrifuge (in particular, the centrifugal direction from a rotation center O to a joint portion with the first transfer tube 21 of the blood collection bag 11) will be denoted by “θ.” The inclination angle θ of the second portion 21b is set such that a distance of the second portion 21b from the rotation center O increases from the first clamp 71 side to a point Q (joint point between the first and second portions 21a and 21b). For example, if the distance between the rotation center O and the point Q (or the distance between the rotation center O and the first transfer tube 21 during the centrifuge) is 150 mm, the inclination angle θ is set to “0°≦θ≦90°,” and preferably, “10°.”

Since the second portion 21b of the first transfer tube 21 held by the first guide path 61 of the cassette 50 extends as described above, the distance between the rotation center O and the first transfer tube 21 during the centrifuge increases across the first transfer tube 21 toward the blood collection bag 11 side. Therefore, in FIG. 3, a length relationship is set to “L1<L2.”

It is noted that the second portion 21b of the first transfer tube 21 may be curved or bent if the first transfer tube 21 is held such that the distance between the rotation center O and the first transfer tube 21 during the centrifuge increases toward the blood collection bag 11 side across the length between a position of the first clamp 71 in the first transfer tube 21 and the blood collection bag 11. In this case, the second portion 21b may be formed in an arc shape.

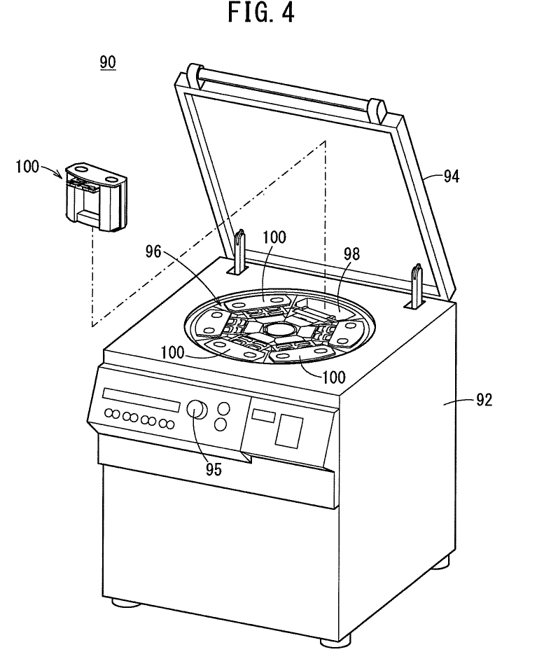

When the blood bag system 10 configured as described above is in use, the blood bag system 10 may be installed, for example, in the centrifuge/transfer apparatus 90 illustrated in FIG. 4 after a blood collection process, and a centrifuge process and a separation/transfer process may be performed using the centrifuge/transfer apparatus 90. That is, the centrifuge/transfer apparatus 90 may perform a centrifuge process for centrifuging whole blood stored in the blood collection bag 11 into three layers including plasma, a buffy coat, and concentrated red blood cells and a separation/transfer process for transferring the plasma to the plasma bag 12, transferring concentrated red blood cells to the third bag 13, and leaving the buffy coat in the blood collection bag 11.

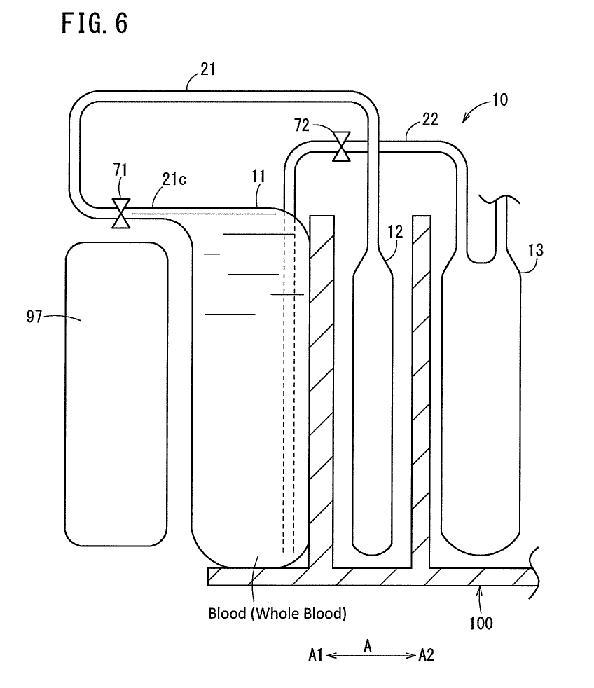

As illustrated in FIG. 4, the centrifuge/transfer apparatus 90 having a box shape includes an apparatus body 92, a lid 94 provided openably/closably on an upper face, an internal centrifugal drum 96, six unit insert holes 98 provided with an equal interval (60°) inside the centrifugal drum 96, six insert units 100 inserted into each of unit insert holes 98, and six plungers 97 (refer to FIG. 6 and the like) provided in a center and advanceable/retreatable in a rotational radial direction with respect to each insert unit 100. The centrifuge/transfer apparatus 90 is operated in response to a manipulation from a manipulation unit 95 provided in the front face and is controlled using a microcomputer (not illustrated).

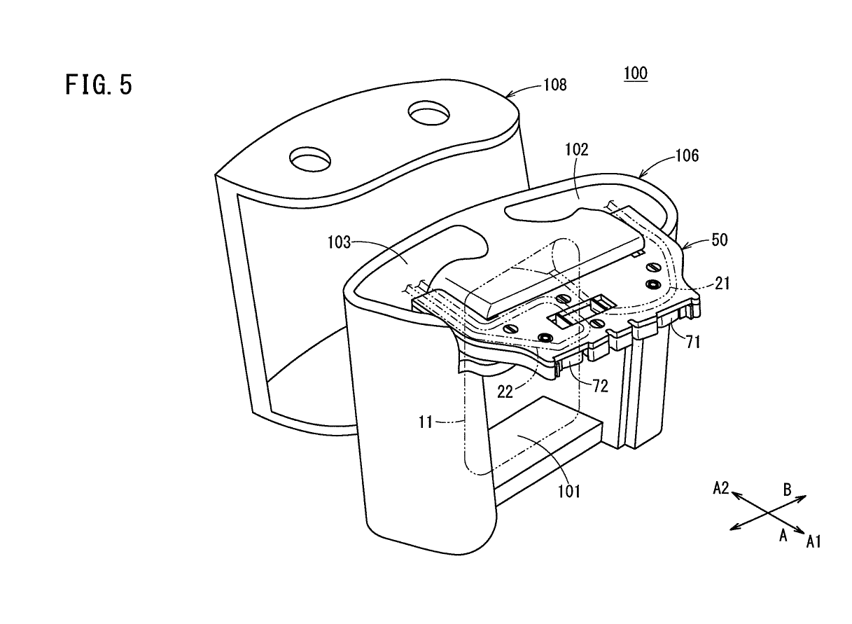

FIG. 5 is an exploded perspective view illustrating the insert unit 100. While the insert unit 100 is inserted into the unit insert hole 98, an arrow direction A of FIG. 5 indicates a radial direction of the centrifugal drum 96. In particular, an arrow direction A2 indicates a centrifugal direction during the centrifuge, and an arrow direction B indicates a circumferential direction of the centrifugal drum 96.

The insert unit 100 has a unit body 106 and a cover body 108. The unit body 106 includes a first chamber 101 opened to the inner diameter side and second and third chambers 102 and 103 opened to an upper side. The cassette 50 is detachably installed in the unit body 106. While the cassette 50 is installed in the unit body 106, the cassette 50 protrudes in an inner diameter direction Al in an upper portion of the first chamber 101.

While the cassette 50 holding the blood collection bag 11 and the first and second transfer tubes 21 and 22 is installed in the insert unit 100, as indicated by a virtual line of FIG. 5, an upper portion of the blood collection bag 11 is bent approximately perpendicularly, and a main body of the blood collection bag 11 is stored in the first chamber 101 of the unit body 106. In this case, the blood collection bag 11 is hung and supported by the cassette 50 and is stored in the first chamber 101 while a thickness direction of the main body is laid in a direction A, that is, the centrifugal direction.

The cover body 108 is a cover installed from an outer side surface to the unit body 106 to cover an outer side surface, an upper surface, and a lower surface of the unit body 106 and reliably hold the blood bag system 10 installed in the unit body 106.

The blood bag system 10 according to this embodiment is basically formed as described above. Next, the effects and advantages thereof will be described.

A process of collecting blood from a donor using the blood bag system 10 of FIG. 1, separating the collected blood into three layers including plasma, a buffy coat, and concentrated red blood cells, and separately storing each component in each bag may be performed, for example, in the following sequence.

First, a blood collection process for puncturing a donor's skin with a blood collection needle 30 and collecting blood from the donor is performed. In the blood collection process, only a predetermined amount of initial droplet of blood (initial flow) is stored in an initial flow blood bag 36 before collection of blood into the blood collection bag 11. In this case, the seal member 20 is closed (initial state), and the clamp 34 is opened. As a result, it is possible to obstruct an initial flow from being directed to the blood collection tube 16 side, that is, the blood collection bag 11 side introduces the initial flow into the initial flow blood bag 36 via the blood collection tube 28, the branching connector 26, and the branching tube 32.

Then, a blood sampling tube (not illustrated) is installed in the sampling port 38 to collect the initial flow in the blood sampling tube. The collected initial flow is used as test blood. It is noted that a portion from the branching connector 26 to the sampling port 38 may be omitted depending on a use.

As the collection of the initial flow is finished, the branching tube 32 is closed with a clamp 34, and a breaking manipulation is performed for the seal member 20 to open a flow path of the blood collection tube 16. In this case, the clamp 18 is opened. Then, the donor's blood is input to the blood collection bag 11 via the blood collection tubes 28 and 16 in this order. It is noted that each flow path of the first and second transfer tubes 21 and 22 is preferably closed by holding the blood collection bag 11 and the first and second transfer tubes 21 and 22 with the cassette 50 and closing the first and second clamps 71 and 72 before collection of blood into the blood collection bag 11.

As a predetermined amount of blood is collected and stored in the blood collection bag 11, the blood collection tube 16 is closed with a clamp 18 in order to prevent a leakage of blood stored in the blood collection bag 11. In addition, the blood collection tube 16 is welded and sealed with a tube sealer and the like, and the blood collection tube 16 is then cut out at the sealed portion. In the following description, out of the entire blood bag system 10 of FIG. 1, a portion closer to the blood collection bag side than the cut-out portion of the blood collection tube 16 will also be referred to as a “blood bag system 10.”

Then, the blood bag system 10 is installed in the centrifuge/transfer apparatus 90 (refer to FIG. 4), so that the whole blood collected in the blood collection bag 11 using the centrifuge/transfer apparatus 90 is separated into plasma, a buffy coat, and concentrated red blood cells, each of which is stored in a predetermined bag.

In installation of the blood bag system 10 into the centrifuge/transfer apparatus 90, the cassette 50 which holds the blood collection bag 11 and the first and second transfer tubes 21 and 22 is installed in the unit body 106 of the insert unit 100. As a result, the blood collection bag 11 hung and supported by the cassette 50 is stored in the first chamber 101 of the unit body 106 (refer to FIG. 5). The plasma bag 12 and the first transfer tube 21 are stored, for example, in the second chamber 102. The third bag 13 and the second transfer tube 22 are stored, for example, in the third chamber 103. Other portions (such as the filter 40 and the fourth bag 14) are stored in, for example, in the second chamber 102 or the third chamber 103.

It is noted that, as a modification of the unit body 106, the second chamber 102 and the third chamber 103 may be connected to form a single storing chamber. In addition, as a modification of the cover body 108, a separate storing chamber may also be further provided. In this case, other portions of the blood bag system 10 described above may be stored in this separate storing chamber.

As the blood bag system 10 is installed and stored in the unit body 106, the cover body 108 is installed in the unit body 106 to make the insert unit 100 in an assembly state.

Then, the insert unit 100 storing the blood bag system 10 is inserted into the unit insert hole 98 of the centrifuge/transfer apparatus 90. Basically, six insert units 100 are installed in the centrifuge/transfer apparatus 90. However, as long as balance is maintained, five or less insert units 100 (preferably, three or two insert units 100 with an equal interval) may also be installed.

Then, the centrifuge process and the separation/transfer process are automatically performed by manipulating the manipulation unit 95 after closing the lid 94 of the centrifuge/transfer apparatus 90. Here, FIG. 6 is a schematic diagram illustrating a state of the blood bag system 10 before the centrifuge process (before applying a centrifugal force). The first and second clamps 71 and 72 are closed in advance. As a result, each flow path of the first and second transfer tubes 21 and 22 is closed.

Since the blood preservation solution having a blood anticoagulant property is stored in the blood collection bag 11 in advance as described above, the blood preservation solution is input to a portion 21c of the first transfer tube 21 between the first clamp 71 and the blood collection bag 11. As a result, after blood is collected in the blood collection bag 11, apart of the blood in the blood collection bag 11 before centrifuge may diffuse to the additive solution in the portion 21c of the first transfer tube 21 and move to the portion 21c of the first transfer tube 21.

In the automatic operation of the centrifuge/transfer apparatus 90, first, the centrifuge process is performed by rotating the centrifugal drum 96.

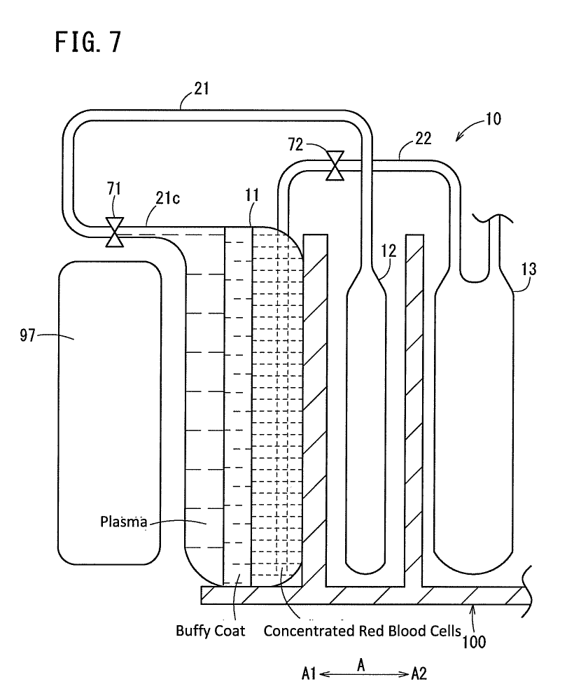

In the centrifuge process, the whole blood stored in the blood collection bag 11 receives a centrifugal force as illustrated in FIG. 7. Therefore, concentrated red blood cells as a high specific gravity component move to a radially outer direction A2, plasma as a low specific gravity component moves to a radially inner direction A1, and a buffy coat as an intermediate specific gravity component moves to a middle position thereof, so that they are separated into three layers.

In this case, in the blood bag system 10 according to this embodiment, (the first guide path 61 of) the cassette 50 holds the first transfer tube 21 such that a distance between a rotation center O of the centrifuge and the first transfer tube 21 increases toward the blood collection bag 11 side across the length between the flow path close position (position of the first clamp 71) of the first transfer tube 21 and the blood collection bag 11 as illustrated in FIG. 3. For this reason, in the centrifuge process, blood cells input to the aforementioned portion 21c of the first transfer tube 21 receive a centrifugal force and are returned to the blood collection bag 11. As a result, it is possible to almost or perfectly remove blood cells from the aforementioned portion 21c of the first transfer tube 21.

A process of the centrifuge/transfer apparatus 90 advances to the separation/transfer process after the centrifuge process. In the separation/transfer process, while a centrifugal force is applied to the blood collection bag 11 by maintaining rotation of the centrifugal drum 96, the first and second clamps 71 and 72 are opened by operating first and second clamping driving units, respectively (not illustrated). As a result, each flow path of the first and second transfer tubes 21 and 22 is opened.

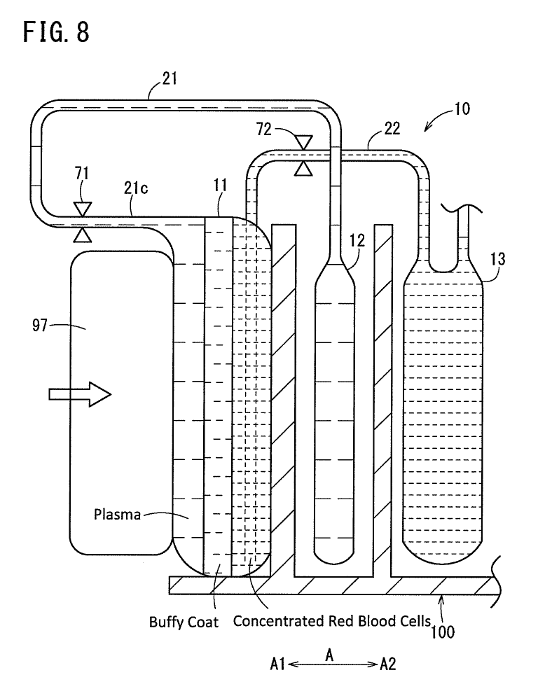

Then, as illustrated in FIG. 8, a plunger 97 is displaced in a centrifugal direction, that is, in a radially outer direction A2 to press the blood collection bag 11. The blood collection bag 11 is plunged between the plunger 97 and the wall so that its volume is reduced. In this case, the first transfer tube 21 is directed to a radially inner side. Therefore, the plasma positioned in the radially innermost side flows from the blood collection bag 11 to the first transfer tube 21 and is input to the plasma bag 12 through the first transfer tube 21.

In this case, as described above, blood cells are almost or perfectly removed from the portion 21c of the first transfer tube 21 in the separation/transfer process. For this reason, when plasma is transferred from the blood collection bag 11 to the plasma bag 12, blood cells are not (or difficultly if any) transferred together. Therefore, it is possible to prevent or suppress blood cells from being mixed into the plasma bag 12.

Meanwhile, since the second transfer tube 22 is connected to the radially outer side of the blood collection bag 11, the concentrated red blood cells positioned in the radially outermost side are output to the second transfer tube 22 from the blood collection bag 11 and are input to the third bag 13 through the second transfer tube 22.

If a microcomputer (not illustrated) detects that plasma is transferred from the first transfer tube 21, and the plunger 97 reaches a predetermined position, the first clamp 71 is closed to shut the flow path of the first transfer tube 21. As a result, red blood cells are input only to the middle of the first transfer tube 21. Therefore, it is possible to obstruct red blood cells from flowing to the plasma bag 12.

Similarly, if an optical sensor (not illustrated) detects that red blood cells are transferred from the second transfer tube 22, and a separation surface between plasma and a buffy coat reaches a predetermined position, the second clamp 72 is closed to shut the flow path of the second transfer tube 22. As a result, red blood cells are input only to the middle of the second transfer tube 22. Therefore, it is possible to obstruct red blood cells from flowing to the third bag 13. The plunger 97 stops when both the first and second clamps 71 and 72 are closed.

As the separation/transfer process described above is terminated, the blood bag system 10 is extracted from the insert unit 100. In addition, the first and second transfer tubes 21 and 22 of the blood bag system 10 are welded and sealed with a tube sealer and the like, and each bag is then removed by cutting them. It is noted that concentrated red blood cells temporarily stored in the third bag 13 are then filtered through the filter 40 to remove leukocyte. The concentrated red blood cells with leukocyte being removed are stored and preserved in the fourth bag 14.

As described above, in the blood bag system 10 according to this embodiment, the cassette 50 of the tube holder holds the first transfer tube 21 such that the distance between the rotation center O of the centrifuge and the first transfer tube 21 increases toward the plasma bag 12 side across the length between the flow path close position of the first transfer tube 21 and the plasma bag 12.

In this configuration, even when a part of blood in the blood collection bag 11 moves to the first transfer tube 21 after blood collection, blood cells input to the first transfer tube 21 in the centrifuge process are almost or perfectly returned to the blood collection bag 11 by virtue of a centrifugal force. Therefore, in the process of transferring plasma obtained through the centrifuge to the plasma bag 12, it is possible to prevent or suppress unnecessary blood cells from being mixed into the plasma bag 12. Therefore, it is possible manufacture plasma derivatives with an excellent appearance quality.

In this embodiment, the cassette 50 holds the first transfer tube 21 such that the first transfer tube 21 has the first portion 21a that extends in the centrifugal direction of the centrifuge and is connected to the plasma bag 12 and the second portion 21b that extends perpendicularly to the centrifugal direction and reaches the first clamp 71. In this configuration, it is possible to prevent or suppress blood cells from being mixed into the plasma bag 12 as described above. In addition, it is possible to hold the first transfer tube 21 in a reasonable shape in terms of a relationship between a position of the first clamp 71 and a holding position of the blood collection bag 11.

In this embodiment, the second portion 21b of the first transfer tube 21 has a straight line shape. For this reason, it is possible to simplify a structure of the portion for holding the first transfer tube 21 in the cassette 50 and facilitate installation of the first transfer tube 21 into the cassette 50.

In the aforementioned embodiment, the cassette 50 illustrated as an example of the configuration of the tube holder is detachably installed in the unit body 106 of the insert unit 100. However, the configuration of the tube holder is not limited thereto. For example, the tube holder may be installed as a part of the unit body 106 and may be provided undetachably from the unit body 106.

The cassette 50 illustrated as an example of the configuration of the tube holder includes the cassette body 52 and the lid body 54, and the first and second transfer tubes 21 and 22 are interposed therebetween. However, a configuration of the tube holder is not limited thereto. For example, the tube holder may be provided with a tunnel-shaped guide path for holding the first and second transfer tubes 21 and 22 on one side surface to expose the first and second transfer tubes 21 and 22 held in the guide path.

Although the tube holder has a configuration of holding both the first and second transfer tubes 21 and 22, the invention is not limited thereto. Instead, only the first transfer tube 21 may be held.

Although whole blood is centrifuged into three components including plasma, a buffy coat, and concentrated red blood cells in the aforementioned embodiment, the invention is not limited thereto. For example, blood obtained by removing leukocyte from whole blood in advance may be centrifuged into three components including a low specific gravity component, an intermediate specific gravity component, and a high specific gravity component, that is, plasma (plasma obtained by removing leukocyte), concentrated platelets (concentrated platelets obtained by removing leukocyte), and concentrated red blood cells (concentrated red blood cells obtained by removing leukocyte).

While the present invention has been described above by showing a preferred embodiment thereof, the invention is not to be restricted to the embodiment described above, and, naturally, various modification or alterations may be possible within the spirit and scope of the invention.

### REFERENCE SIGNS LIST

10 blood bag system

11 first bag (blood collection bag)

12 second bag (plasma bag)

21 first transfer tube

21a first portion

21b second portion

22 second transfer tube

50 cassette

61 first guide path

62 second guide path

71 first clamp

72 second clamp

90 centrifuge/transfer apparatus

1. A blood bag system comprising:
a first bag configured to preserve whole blood or a blood component;
a tube configured to transfer a component obtained by centrifuging a content of the bag to an outer side of the bag; and
a tube holder configured to hold at least a part of the tube,
wherein a clamp is configured to close and open a flow path while the tube is held by the tube holder, and
the tube holder holds the tube such that a distance between a rotation center in application of a centrifugal force and the tube increases toward the bag across the length of the tube between position of the clamp on the tube and a position of the bag.

2. The blood bag system according to claim 1, wherein the tube holder holds the tube such that the tube has a first portion that extends in a centrifugal direction and is connected to the bag and a second portion that extends perpendicularly to the centrifugal direction and reaches the flow path close position.

3. The blood bag system according to claim 2, wherein the second portion has a straight line shape.

4. The blood bag system according to claim 2, wherein the second portion is curved or bent.

5. The blood bag system according to claim 1, further comprising:
a second bag configured to store the component transferred through the tube; and
a third bag configured to store a high specific gravity component.

6. The blood bag system according to claim 1, wherein the component transferred from the first bag to an outer side of the bag is plasma.

7. A tube holder having a guide path configured to hold at least a part of a tube for transferring a component obtained by centrifuging a content of a bag to an outer side of the bag,
wherein the tube is configured to close and open a flow path while the tube is held by the tube holder, and
the guide path holds the tube such that a distance between a rotation center of a centrifugal force and the tube increases toward the bag side across a length from a flow path close position of the tube and the bag.
